(19) United States
(12) Patent Application Publication
(10) Pub. No.: US 2008/0156320 A1
(43) Pub. Date: Jul. 3, 2008

(54) ULTRASONIC NEBULIZER AND METHOD FOR ATOMIZING LIQUID

(76) Inventors: Thomas Low, Belmont, CA (US); Pablo Garcia, Menlo Park, CA (US); Jeff Shimon, Sunnyvale, CA (US); Paul T. Kotnik, Commerce Township, MI (US); David A. Ross, Columbiaville, MI (US)

Correspondence Address: CANTOR COLBURN, LLP, 20 Church Street, 22nd Floor, Hartford, CT, 06103, US

(21) Appl. No.: 11/619,445

(22) Filed: Jan. 3, 2007

(51) Int. Cl. A61M 11/00

(52) U.S. Cl. 128/200.16

(57) ABSTRACT

A nebulizer and a method for atomizing liquid are provided. The method includes directing liquid from a first chamber to a second chamber in a housing. The second chamber is configured to form standing pressure waves therein when the liquid therein is vibrated. The method further includes vibrating the liquid in the second chamber utilizing a piezo-electric device such that standing pressure waves are formed in the second chamber. The method further includes atomizing liquid in the second chamber as the liquid propagates through a meshed screen wherein the meshed screen is disposed a predetermined distance from the piezo-electric device at a high amplitude region of the standing pressure waves and within a focal region of the piezo-electric device.

### BACKGROUND

This application relates to an ultrasonic nebulizer and a method for atomizing a liquid.

U.S. Pat. No. 6,670,034 describes a nebulizer that atomizes a liquid. The nebulizer utilizes a piezoelectric crystal that vibrates a titanium horn. The titanium horn amplifies the vibrations of the piezoelectric crystal and in turn vibrates liquid contacting the titanium horn such that the liquid is subsequently atomized. A disadvantage with the foregoing design is that the titanium horn is a relatively expensive component.

Accordingly, the inventors herein have recognized that it would be advantageous to have a nebulizer that utilizes the liquid as the amplification medium instead of the titanium horn.

### SUMMARY OF THE INVENTION

A nebulizer in accordance with an exemplary embodiment is provided. The nebulizer includes a housing having a first chamber and a second chamber. The first chamber is configured to hold a liquid therein. The second chamber is configured to receive the liquid from the first chamber. The second chamber is configured to form standing pressure waves therein when the liquid therein is vibrated. The nebulizer further includes a piezo-electric device disposed in the second chamber. The piezo-electric device is configured to vibrate the liquid in the second chamber such that standing pressure waves are formed in the second chamber. The nebulizer further includes a meshed screen disposed at a predetermined distance from the piezo-electric device at a high amplitude region of the standing pressure waves and within a focal region of the piezo-electric device such that liquid in the second reservoir is atomized as the liquid propagates through the meshed screen.

A method for atomizing a liquid in accordance with another exemplary embodiment is provided. The method includes directing liquid from a first chamber to a second chamber in a housing. The second chamber is configured to form standing pressure waves therein when the liquid therein is vibrated. The method further includes vibrating the liquid in the second chamber utilizing a piezo-electric device such that standing pressure waves are formed in the second chamber. The method further includes atomizing liquid in the second chamber as the liquid propagates through a meshed screen wherein the meshed screen is disposed a predetermined distance from the piezo-electric device at a high amplitude region of the standing pressure waves and within a focal region of the piezo-electric device.

### BRIEF DESCRIPTION OF THE DRAWINGS

FIG. 1 is a schematic of a system for atomizing a liquid having a nebulizer in accordance with an exemplary embodiment;

FIG. 2 is a graph of a simulated pressure waveform for a 6 mm diameter piezo-electric device in a nebulizer operating at 2.38 MHz;

FIG. 3 is a graph of a simulated pressure waveform for a 10 mm diameter piezo-electric device in a nebulizer operating at 2.38 MHz;

FIG. 4 is a graph of simulated standing pressure waves in the nebulizer of FIG. 1;

FIG. 5 is a top view a meshed screen utilized in the nebulizer of FIG. 1 illustrating only a portion of the orifices extending through the meshed screen;

FIG. 6 is an enlarged cross-sectional schematic of a portion of the meshed screen utilized in the nebulizer of FIG. 1; and

FIG. 7 is a schematic of a system for atomizing a liquid in accordance with another exemplary embodiment.

### DETAILED DESCRIPTION OF EXEMPLARY EMBODIMENTS

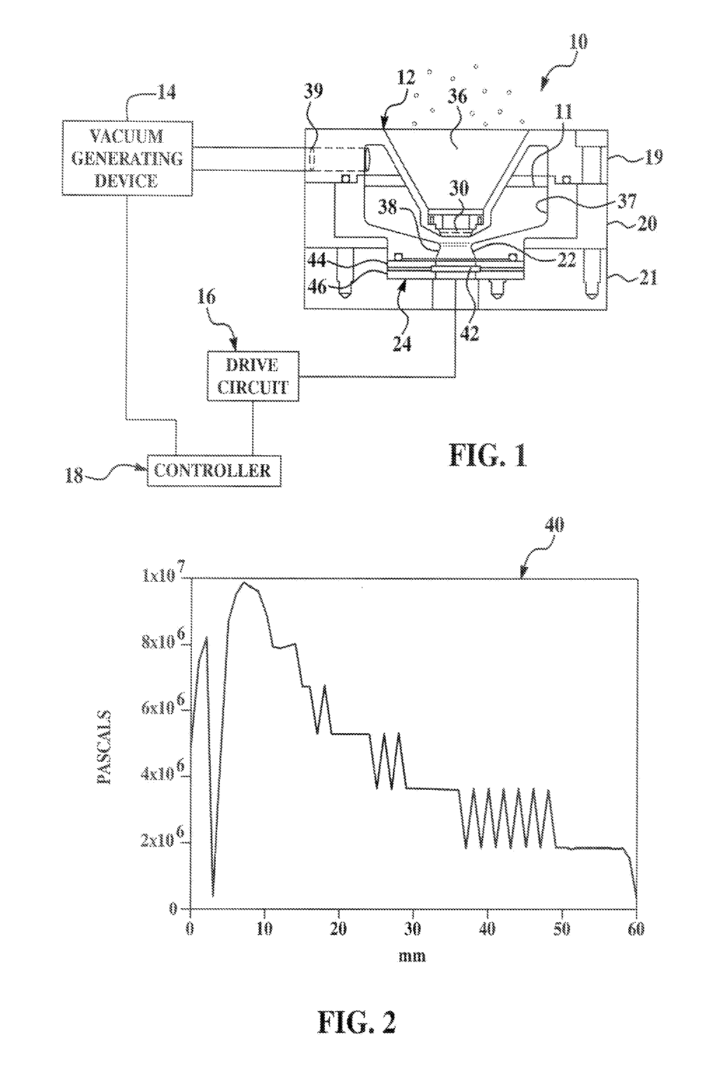

Referring to FIG. 1, a system 10 for atomizing a liquid 11 in a nebulizer 12 in accordance with an exemplary embodiment of the present invention is provided. The term “atomizing” as used herein means to reduce or to separate a liquid into tiny particles or into a fine spray. The system 10 comprises the nebulizer 12, a vacuum generating device 14, a drive circuit 16, and a controller 18.

The nebulizer 12 is provided for atomizing the liquid 11 into tiny particles. The nebulizer 12 includes a housing formed from plates 19, 20, and 21, a piezo-electric device 24, and a meshed screen 30.

The top plate 19 is coupled to a top surface of the plate 20. The bottom plate 21 is coupled to a bottom surface of the plate 20. The plates 19, 20, and 21 can be coupled together using any known fastening means, such as bolts for example. The plate 19 and the plate 20 define chambers 37 and 38 therein. The chambers 37 and 38 are provided for holding the liquid 11 therein. The chamber 38 communicates with the chamber 37. The plate 19 further has a fill port 39 extending therethrough. In one exemplary embodiment, the plates 19, 20, and 21 are rigid plastic plates. Of course in alternative embodiments, the plates 19, 20 and 21 can be constructed from other materials, such as stainless steel for example. Further, the chamber 37 can be replaced by a collapsible bag which holds the liquid therein. The chamber 38 has a relatively small volume that is less than a volume of the chamber 37. In one exemplary embodiment, the chamber 38 has a volume less than 0.3 cc. Of course in alternative embodiments, the chamber 39 can have a volume other than 0.3 cc. The plates 19 and 20 further define a conically shaped outlet nozzle portion 36 that has an aperture extending therethrough which receives atomized liquid from the chamber 38. The outlet nozzle portion 36 directs atomized liquid from the meshed screen 30 to the environment.

The chamber 38 is bounded on one end by the piezo-electric device 24 and on the opposite end by the meshed screen 30. Further, the shape and length of the chamber 38 are configured to form standing pressure waves therein when liquid therein is vibrated and reflected back from the meshed screen 30. In one exemplary embodiment, the meshed screen 30 is placed in a region having a highest acoustic pressure variation or a pressure amplitude in the chamber 38.

The piezo-electric device 24 is provided for generating standing pressure waves in the liquid 11 of the chamber 38. In one exemplary embodiment, the piezo-electric device 24 include a piezo-electric member 42 disposed between plates 44 and 46. The piezo-electric member 42 is electrically coupled to the drive circuit 16 and vibrates in response to a control signal from the drive circuit 16. During operation, in one exemplary embodiment, the piezo-electric device 24 generates standing pressure waves having a frequency in a frequency range of 1.67-2.44 MHz. Of course, in alternative embodiments, the piezo-electric device 24 could generate standing pressure waves having a frequency less than 1.67 MHz or greater than 2.44 MHz.

The meshed screen 30 is provided to atomize liquid pressure waves contacting the meshed screen 30. The meshed screen 30 is disposed at a predetermined distance from the piezo-electric device 24 at a high amplitude region of the standing pressure waves and within an acoustic focal region of the piezo-electric device 24. In particular, the operating frequency of the piezo-electric device 24 and the dimensions of the chamber 38 are chosen so as to place the meshed screen 30 in the acoustic focal region. In one exemplary embodiment, the predetermined distance between the piezo-electric device 24 and the meshed screen 30 is at least one wavelength of one of the standing pressure waves. Further, the amplitude of the pressure waves is greater than or equal to a desired amplitude in the acoustic focal region to facilitate atomizaiton of the liquid 11 by the meshed screen 30. Liquid in the chamber 38 is atomized as the liquid propagates through the meshed screen 30 at relatively high rates and at relatively low power levels of the piezo-electric device 24. In particular, the standing pressure waves contact the meshed screen 30 to atomize the liquid 11 propagating through the meshed screen 30 into the outlet nozzle 36. It is noted that the meshed screen 30 and the piezo-electric device 24 are removable from the nebulizer 12. When the meshed screen 30 and the piezo-electric device 24 are removed from the nebulizer 12, the chambers 37 and 38 may be readily rinsed.

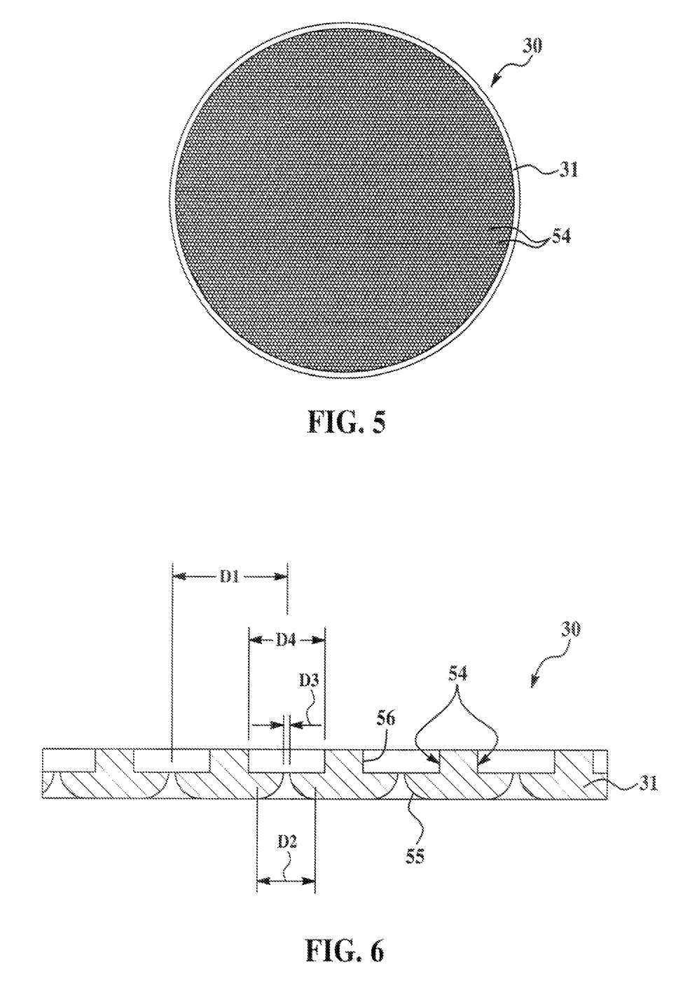

Referring to FIG. 5, a top view of the meshed screen 30 showing only a portion of the plurality of orifices 54 extending through the meshed screen 30 is illustrated. In one exemplary embodiment, the meshed screen 30 has a body portion 31 with a diameter of 0.6 millimeters and a thickness of 25 microns. Of course in other exemplary embodiments, the meshed screen 30 could have a body portion 31 with a diameter greater than 6.0 millimeters or less than 6.0 millimeters and a thickness greater than 25 microns or less than 25 microns. Further, the orifices 54 have a center-to-center spacing (D1) of 60 microns and extend 13 microns into the meshed screen. Of course in other exemplary embodiments, the orifices 54 could have a center-to-center spacing of greater than 60 microns or less than 60 microns and extend greater than 13 microns or less than 13 microns in the meshed screen 30. Referring to FIG. 6, an enlarged cross-sectional view of a portion of the meshed screen 30 is illustrated. Each of the orifices 54 has a bell-shaped orifice portion 55 communicating with a hexagonal-shaped orifice portion 56. In one exemplary embodiment, a maximum diameter (D2) of the orifice portion 55 is 33 microns and a minimum diameter (D3) of the orifice portion 55 is 3 microns. Of course in alternative embodiments, the diameters (D2) and (D3) could be modified from these exemplary values. Further, in one exemplary embodiment, each hexagonal-shaped orifice portion 56 has a diameter (D4) of 40 microns and extends 12 millimeters into the meshed screen 30. Of course in alternative embodiments, the diameter (D3) orifice portion 56 could be modified from the exemplary diameter and the depth of the orifice portion 56 could be modified from the exemplary depth. Pressure waves generated in the liquid 11 cause the liquid 11 to be ejected through the orifices 54, and to form tiny particles of a desired size. The orifice portions 55 are disposed in a facing relationship with respect to the piezo-electric device 24. It should be further noted that the meshed screen 30 could have a plurality of alternative shapes, other than being generally circular shaped.

The controller 18 is provided to control the operation of the drive circuit 16 that energizes the piezo-electric device 24. In particular, the controller 18 generates command signals that are received by the drive circuit 16. The command signals induce the drive circuit 16 to generate drive signals to induce the piezo-electric device 24 to oscillate or vibrate. The controller 18 is further provided to generate command signals for controlling operation of the vacuum generating device 14.

The vacuum generating device 14 is provided to generate a negative pressure in an air space of the chamber 37 above the liquid therein. The negative pressure in the air space of the chamber 37 causes liquid in the chamber 38 to be pulled away from the meshed screen 30 such that meshed screen 30 is not submerged in the liquid of chamber 38 which assists in atomizing liquid at the meshed screen 30.

Referring to FIG. 2, a graph 40 shows a simulated pressure waveform associated with a piezo-electric device. The graph 40 was utilized to determine an acoustic focal region for a piezo-electric device having a predetermined size and operating frequency. In particular, to obtain the graph 40, the following simulated parameters were utilized: (i) a disk shaped piezo-electric device having a 6 mm diameter, and (ii) the piezo-electric device operating at a frequency of 2.38 MHz. The y-axis of FIG. 2 depicts the amplitude, and the x-axis depicts a distance from the piezo-electric device in millimeters. FIG. 2 shows that the acoustic focal region for the standing pressure waves occurs in a range of 5-12 mm from the piezo-electric device.

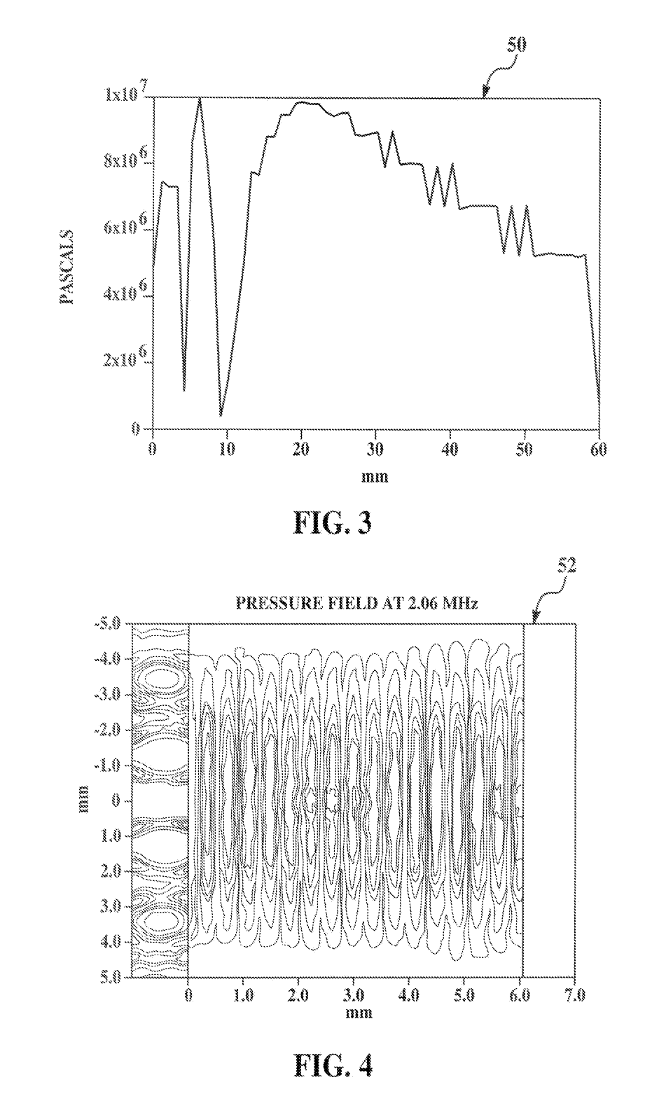

Referring to FIG. 3, a graph 50 shows another simulated pressure waveform of a piezo-electric device. The graph 40 was utilized to determine an acoustic focal region for another piezo-electric device having a predetermined size and operating frequency. In particular, to obtain the graph 50, the following simulated parameters were utilized: (i) a disk shaped piezo-electric device having a 10 mm diameter, and (ii) the piezo-electric device operating at a frequency of 2.38 MHz. The y-axis of FIG. 3 depicts the amplitude, and the x-axis depicts a distance from the piezo-electric device in millimeters. FIG. 3 shows that the acoustic focal region for the standing pressure waves occurs in a range of 15-25 mm from the piezo-electric device.

Referring to FIG. 4, a graph 52 of standing pressure waves in the nebulizer in a region between a piezo-electric device and a meshed screen is illustrated. To obtain graph 52, the following simulated parameters were utilized: (i) a piezo-electric device 24 placed at 6.2 mm distance from the meshed screen 30, and (ii) the piezo-electric device operating at a frequency of 2.06 MHz. The x-axis of the graph indicates a distance from the piezo-electric device, and the y-axis indicates a radial distance from the piezo-electric device. The dark central regions in a range of 6.0-6.2 mm along the x-axis indicate a maximum amplitude of pressure waves, occurs in this range.

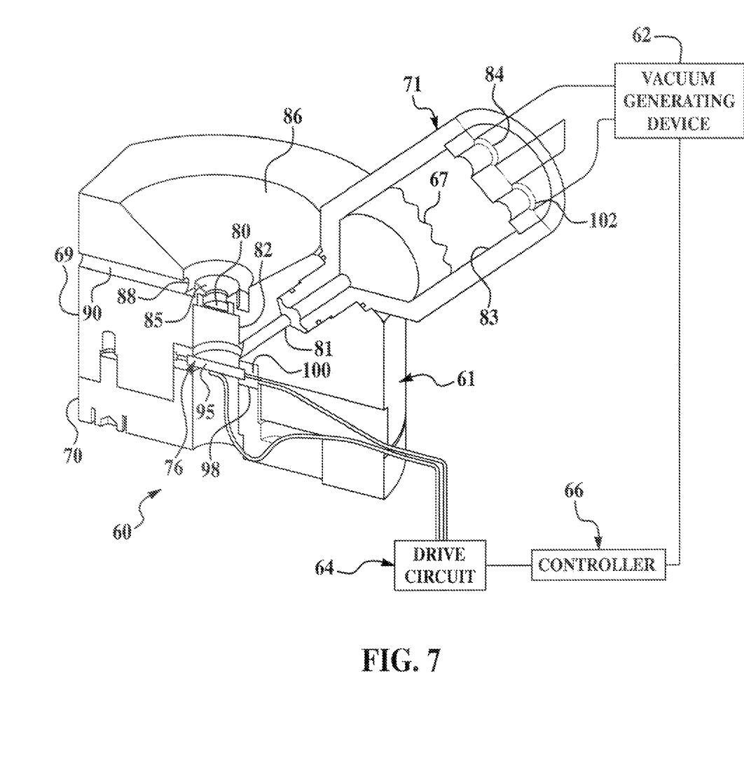

Referring to FIG. 7, a system 60 for atomizing a liquid 67 in a nebulizer 61 in accordance with another exemplary embodiment is provided. The system 60 comprises the nebulizer 61, a vacuum generating device 62, a drive circuit 64, and a controller 66.

The nebulizer 61 is provided for atomizing the liquid 67 into tiny particles. The nebulizer 61 comprises a housing formed by plates 69 and 70, a container 71, a piezo-electric device 76, and a meshed screen 80.

The top plate 69 is coupled to a top surface of the bottom plate 70. The top plate 69 includes an aperture 81 extending from an outer surface thereof to a container chamber 82 defined by the top plate 69. A portion of the aperture 81 is configured to receive a corresponding portion of the container 71. The container 71 has a chamber 83 therein configured to store a liquid therein. The container 71 also has fill ports 84, 102 extending therethrough. A liquid in the chamber 83 flows through the aperture 81 of the top plate 69 into the chamber 82 defined by the top plate 69. In one exemplary embodiment, the plates 69, 70 can be constructed from other materials such as stainless steel for example. In an alternative embodiment, the container 71 can be replaced by a collapsible bag which holds a liquid therein. The chamber 82 has relatively small volume that is less of a volume of the chamber 83. The plate 69 further defines conically shaped nozzle portions 85, 86. The conically shaped nozzle portion 85 has an aperture extending therethrough which receives atomized liquid form the chamber 82. A drain groove 88 extends around the conically shaped nozzle portion 85 between the conically shaped nozzle portion 85 and the conically shaped nozzle portion 86. The drain grove 88 is provided to route liquid adhering to the nozzle portion 86 into a drain aperture 90 which extends through the plate 69 to an outer surface of the plate 69. The nozzle portion 85 and 86 direct atomized liquid outwardly from the nebulizer 61.

The piezo-electric device 76 is provided to generate standing pressure waves in the liquid 67 of the chamber 82. In one exemplary embodiment, the piezo-electric device 76 include a piezo-electric member 95 disposed between plates 98 and 100. The piezo-electric member 95 is electrically coupled to the drive circuit 64 and vibrates in response to a control signal from the drive circuit 64. During operation, in one exemplary embodiment, the piezo-electric device 76 generates standing pressure waves having a frequency in a frequency range of 1.67-2.44 MHz. Of course, in alternative embodiments, the piezo-electric device 76 could generate pressure waves having a frequency less than 1.67 MHz or greater than 2.44 MHz.

The meshed screen 80 is provided to atomize liquid pressure waves contacting the meshed screen 80. The meshed screen 80 is disposed at a predetermined distance from the piezo-electric device 76 at a high amplitude region of the standing pressure waves and within an acoustic focal region of the piezo-electric device 76. In particular, the operating frequency of the piezo-electric device 76 and the dimensions of the chamber 82 are chosen so as to place the meshed screen 80 in the acoustic focal region. In one exemplary embodiment, the predetermined distance between the piezo-electric device 76 and the meshed screen 80 is at least one wavelength of one of the standing pressure waves. Further, the amplitude of the pressure waves is greater than or equal to a desired amplitude in the acoustic focal region to facilitate atomization of the liquid 67 by the meshed screen 80. Liquid in the chamber 82 is atomized as the liquid propagates through the meshed screen 80 at relatively high rates and at relatively low power levels of the piezo-electric device 76. In particular, the standing pressure waves contact the meshed screen 80 to atomize the liquid 67 propagating through the meshed screen 80 into the outlet nozzle portions 85 and 86. It is noted that the meshed screen 80 and the piezo-electric device 76 are removable from the nebulizer 61. When the meshed screen 80 and the piezo-electric device 76 are removed from the nebulizer 61, the chamber 82 may be readily rinsed.

The controller 66 is provided to control the operation of the drive circuit 64 that energizes the piezo-electric device 76. In particular, the controller 66 generates command signal that are received by the drive circuit 64. The command signals induce the drive circuit 64 to generate drive signals to induce the piezo-electric device 76 to oscillate or vibrate. The controller 66 is further provided to generate command signals for controlling operation of the vacuum generating device 62.

The vacuum generating device 62 is provided to generate negative pressure in an air space of the chamber 83 above the liquid therein. The negative pressure in the air space of the chamber 83 causes liquid in the chamber 82 to be pulled away from the meshed screen 80 such that meshed screen 80 is not submerged in the liquid of chamber 82 which assists in atomizing liquid at the meshed screen 80.

In general, the exemplary embodiments of this application can achieve an atomized liquid particle size of less than or equal to 5 μm, a nebulization rate of 0.6-1 mL/min, a battery life of at least 25 medicinal atomization treatments, an angular range of operation that is greater than or equal to 45 degrees off a nominal vertical, and a residual volume of less than or equal to 0.3 mL. Further, the piezo-electric devices 24, 76 of the nebulizers 12, 61, respectively, utilize less than 1.5 watts of electrical power and are oscillated at a frequency in a frequency range of 1.67-2.44 MHz to generate the pressure waves. Also, at least 0.7 milliliters of the liquid is atomized per minute.

The nebulizers and the methods for atomizing a liquid provide substantial advantages over other systems and methods. In particular, the nebulizers have a technical effect of utilizing a piezo-electric transducer and a meshed screen to generate standing pressure waves to amplify vibrations of the piezo-electric device in a chamber such that a meshed screen in the chamber atomizes liquid while utilizing a relatively low amount of electrical power.

While the invention has been described with reference to exemplary embodiments, it will be understood by those skilled in the art that various changes may be made and equivalent elements may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments contemplated for carrying out this invention, but that the invention will include all embodiments falling within the scope of the present application.

What is claimed is:

1. A nebulizer, comprising:
a housing having a first chamber and a second chamber, the first chamber configured to hold a liquid therein, the second chamber configured to receive the liquid from the first chamber, the second chamber configured to form standing pressure waves therein when the liquid therein is vibrated;
a piezo-electric device disposed in the second chamber, the piezo-electric device configured to vibrate the liquid in the second chamber such that standing pressure waves are formed in the second chamber; and
a meshed screen disposed at a predetermined distance from the piezo-electric device at a high amplitude region of the standing pressure waves and within a focal region of the piezo-electric device such that liquid in the second reservoir is atomized as the liquid propagates through the meshed screen.

2. The nebulizer of claim 1, wherein the predetermined distance comprises at least one wavelength of one of the standing pressure waves.

3. The nebulizer of claim 1, further comprising a vacuum generating device configured to supply a vacuum in a region in the first chamber above the liquid contained therein.

4. The nebulizer of claim 3, wherein the vacuum is at a vacuum level sufficient to prevent the meshed screen from being submerged in liquid in the second chamber.

5. The nebulizer of claim 1, wherein the piezo-electric device utilizes less than 1.5 watts of electrical power.

6. The nebulizer of claim 1, wherein the piezo-electric device is oscillated at a frequency in a frequency range of 1.67-2.44 MHz to generate the standing pressure waves.

7. The nebulizer of claim 1, wherein at least 0.7 milliliters of the liquid is atomized per minute.

8. A method for atomizing a liquid, comprising:
directing liquid from a first chamber to a second chamber in a housing, the second chamber being configured to form standing pressure waves therein when the liquid therein is vibrated;
vibrating the liquid in the second chamber utilizing a piezo-electric device such that standing pressure waves are formed in the second chamber; and
atomizing liquid in the second chamber as the liquid propagates through a meshed screen wherein the meshed screen is disposed a predetermined distance from the piezo-electric device at a high amplitude region of the standing pressure waves and within a focal region of the piezo-electric device.

9. The method of claim 8, wherein the predetermined distance comprises at least one wavelength of one of the standing pressure waves.

10. The method of claim 8, further comprising applying a vacuum in a region in the first chamber above the liquid contained therein.

11. The method of claim 10, wherein the vacuum is at a vacuum level sufficient to prevent the meshed screen from being submerged in liquid in the second chamber.

12. The method of claim 8, wherein the piezo-electric device utilizes less than 1.5 watts of electrical energy.

13. The method of claim 8, wherein the piezo-electric device is oscillated at a frequency in a frequency range of 1.67-2.44 MHz to generate the standing pressure waves.

14. The method of claim 8, wherein at least 0.7 milliliters of the liquid is atomized per minute.
